(19) United States
(12) Patent Application Publication
(10) Pub. No.: US 2013/0225412 A1
(43) Pub. Date: Aug. 29, 2013

(54) SILICON NANOCARRIER FOR DELIVERY OF DRUG, PESTICIDES AND HERBICIDES , AND FOR WASTE WATER TREATMENT

(71) Applicants: Soroush Sardari Lodriche, Tehran (IR); Saeed Soltani, Tehran (IR); Roghieh Mirzazadeh, Tehran (IR)

(72) Inventors: Soroush Sardari Lodriche, Tehran (IR); Saeed Soltani, Tehran (IR); Roghieh Mirzazadeh, Tehran (IR)

(21) Appl. No.: 13/406,538

(22) Filed: Feb. 28, 2012

(51) Int. Cl. A61K 9/00; A01N 37/22; A01N 33/06; A01N 43/52; A01N 43/653; A61K 31/57; A61K 31/513; A61K 31/496; A61K 31/4196; A61K 31/664; A61K 31/43; A61K 31/616; A61K 38/12; A61K 31/7048; A61K 31/465; A61K 31/702; A61P 31/10; A61P 31/04; A61P 35/00; A01P 3/00; A01P 13/00; B01J 20/22; A01N 25/08

(52) U.S. Cl. 504/322; 504/358; 504/326; 514/394; 514/383; 514/177; 514/274; 514/254.07; 514/110; 514/197; 514/165; 514/253.08; 514/21.1; 514/31; 514/343; 514/61; 424/400; 252/180

(57) ABSTRACT

The various embodiments herein provide a nano silicon carrier as a drug delivery mechanism. The nano silicon carrier comprises a diatom frustules loaded with a drug molecule to be released at the target site. The diatom frustules are of Hannaea arcus and Navicula inflexa species of diatom. The pore size of the diatom frustules is 1 to 100 nm. The diatom frustules are comb-like in structure. The diatom frustules are in the form of powdered diatomaceous earth. The drug delivery mechanism described in the embodiments herein is a controlled release mechanism. The nano silicon carrier described in the embodiments herein is also used for delivery of pesticides and herbicides in plants. The nano silicon carrier described in the embodiments herein is also used in hormone waste water treatment.

### BACKGROUND

1. Technical Field

The embodiments herein generally relate to a field of drug delivery systems and particularly to a drug delivery system using silicon nano vehicle or carrier and more particularly to a drug delivery system using diatoms as drug delivery vehicles or carriers.

2. Description of the Related Art

The desired loading and release properties for drug delivery could be achieved through a selection of appropriate systems and drug molecules. The designing of such new carriers for drug delivery is an important field of research. Nanotechnology as one of the new and latest fields of technology can help us to use naturally occurring structures in designing a new type of drug vehicles using diatoms. The diatoms are unicellular microalgae.

Diatoms belong to Chrysophyta division, Bacillariophaceae class with a cell wall consisting of a siliceous skeleton or Frustule enveloped by an organic case essentially composed of polysaccharides and proteins. Diatoms as a group are very diverse in nature, comprising some 12,000-60,000 reported species and a common diameter of 20-200 microns. Diatoms are divided into two broad groups: centric and pinnate, depending on the symmetry of frustules. All diatoms have, parallel striates or furrows or rows of holes, in the silica arranged normal to the structural axis. The spacing between the adjacent striate is species specific, but typically varies from 0.3 to 2 μm. The line of silica between the striate is called costae. Costae tend to be symmetrically arranged in combs or other space filling patterns.

In the recent years some attempts have been made to use frustules in nanotechnology applications. Some researches in this field are as follows:

Immuno-isolation and immunological bio-encapsulation could benefit from the frustule properties. The key feature of such capsules would be the ability of frustule pores to protect the enclosed tissue from immune rejection while allowing an adequate supply of nutrients and oxygen. One possible drawback could be the immune reactivity of the silica itself.

Microfabrication: diatom frustules have potential use in the production of nanomaterials of constant diameter. For example, using a template synthesis method, it may be possible to arrange these pores to form nanometer sized tubes and/or fibers.

Lithographic applications: using the costae of pinnate diatoms as a mask in the lithographic process led to the formation of parallel array of uniform channels. Use of centric diatoms, for which their exposed surface is resistant to the etching process, leads to a formation of radiating channels.

Magnetized frustules for pinpoint drug delivery: to produce magnetized frustules, diatoms can be cultured in an iron-rich environment, as described by Wee et al. Drugs or vaccines could be loaded into the pores and delivered by a magnetic field manipulation at a target location within the human or animal body.

Nano-formulation for drugs using the diatom skeleton nano-vehicles: Recently investigations have been made to use the specific nanostructure of diatom frustules as nano-vehicles for drugs. Simulations show that diatom frustules could have a regular specificity of a drug release pattern, which would be extremely useful for nano drug delivery, especially in the cancer treatments.

The evaluation of loading molecules or compounds into nano-carrier includes some parameters. Previously, some useful parameters like molecular weight, number and percentage of nitrogen and oxygen atoms, polarizability (cm³), monoisotopic mass (Da), density (g/cm³), nominal mass (Da), surface tension (dyne/cm), index of refraction, molar volume (cm³), molar refractivity (cm³), hydration energies (Kcal/mol), average mass (Da), parachor (cm³) and the log base 10 of parachor (Log P) have been measured. These physio-chemical properties have been previously shown to be best in describing the controlling factors for loading drugs.

But no work has been done in using these diatoms for delivering the drugs efficiently at the target site. Hence the embodiments herein provide the use of diatoms as nano drug delivery carrier for the delivery of drugs at their target sites. Also the embodiments herein provide the use of diatoms in the field of agriculture and wastewater treatment.

The above mentioned shortcomings, disadvantages and problems are addressed herein and which will be understood by reading and studying the following specification.

### OBJECTIVES OF THE EMBODIMENTS

The primary object of the embodiments herein is to provide a novel drug delivery system using diatoms.

Another object of the embodiments herein is to provide a nano drug delivery system using diatom frustules for various applications.

Yet another object of the embodiments herein is to provide a nano drug delivery system made up of siliceous diatomaceous structure for delivering various pharmaceutical drugs in humans and animals.

Yet another object of the embodiments herein is to provide a nano drug delivery system that can be used for delivering agricultural drugs and pesticides.

Yet another object of the embodiments herein is to provide a nano drug delivery system that can be used for delivering anticancer drugs.

Yet another object of the embodiments herein is to provide a nano drug delivery system that can be used for treating the fungal infections in humans.

Yet another object of the embodiments herein is to provide a nano drug delivery system that can be used for the removal of hormonal waste from wastewater and industrial sewage.

These and other objects and advantages of the embodiments herein will become readily apparent from the following detailed description taken in conjunction with the accompanying drawings.

### SUMMARY

The various embodiments herein provide a nano silicon carrier made up of diatom frustules that can be loaded with various drugs and can be taken to a target sit for drug release.

The embodiments herein provide a silicon nano carrier comprising a diatom frustule and a drug. The diatom frustule is in a form of a half cylinder. The diatom frustule has a comb-like structure. The drug is present inside the diatom frustule and is released at a target site. The diatom frustule includes Hannaea arcus and Navicula inflexa. The diatom frustule has a pore size of 1 to 100 nm. The diatom frustule is prepared in the form of a powder. The diatom frustule is a sieved diatomaceous earth. The silicon nano carrier is used as drug delivery system. The drug delivery is a controlled release of the drug delivery.

The drug is selected from a group comprising of acarbose, acetaminophen, acetanilide, acetazolamide, acetylsalicylic acid, allopurinol, alosetron, alphaprodine, aminogluthetimide, amodiaquine, amthamine, aprepitant, aromatase inhibitor, balsalazide, benzamides sulpiride, benomyl, benzylhydrazine, benzylpenicillin, bezafibrate, buclizine, caffeine, carbendazim, carbutamide, carmustine, chlorambucil. A, chlorhexidine, chlorobutanol, ciprofloxacin, clemastine, clobazam, clomethiazole, cyclaradine, cyclopho sphaide, decarbonylase inhibitor, diflunisal, diperodon, diphenhydramine, diphenoxylate, diphenydramine, diphenylbutylpiper, diphenylpyraline, domperidone, dopamine, doxylamine, ephedrine, epinephrine, ethanolamine, etomidate, eugenol, fadrazole, felodipine, flufenamic acid, fluorouracil, flurandrenolone, furazolidone. A, fenbuconazole, gabapentin, gallic acid, histamine, hordenine, hydralazine, hexachlorobenzene, hydroxyzine, hyoscyamine, ibuprofen, indomethacin, isogramine, isoleucine, isoproterexol, ketorolac, khellin, lamotrigine, lansoprazole, lisoleucine, lomustine, lysine, marinol, meperidine, mercaptopurine, mescaline, metformin, methadone, methantheline, methicilin, methoxamine, methyldopahydrazine, metronidazole, metyrapone, misonidazole, mitomycin C, muzolinine, nabilone, nafoxidine, natamycin, natamycin, nicolsamide, nicotine, nifurtimox, nizatidine, nystatin, ofurace, ofloxacin, omeprazole, oxamniquine, oxaprozine, pentamidine, phenacetin, phenmetrazine, phenoxymethylpenicilin, phenphormin, phenylephrine, phosphor amide, polymyxin B sulphate, pramoxine, praziquantal, procaine, progesterone, pyrantel, quinacrine, razoxane, ritodrine, rosiglitazone, roxatidine, salicylamid, selenazofurin, sulfaalazine, sulfacytine, sulfamerazine, sulfamethazine, sulfamethizole, sulfamethoxazole, sulphanilamide, sulinpyrazone, terfenadine, thiabendazole, thiabedazole, thiaisoleucine, thialysine, tiazofurin, ticlopidine, thiophanate-methyl, tilorone, tocamide, tolazamide, triplennamine, valnoctamide, valporic acid, vicuprofen, vigabartin, zidovudine, zopicolone, 4-hydroxyandrostene, 5-Hydroxytryptamine, 3,4-epoxide, 2′3′-Dideoxyinosine, 2′,3′-dideoxy-2′,3′-didehydrocytidine(d4C), 1-(5-hydroxymethyl-2-furyl)-9H-pyrido(3,4-b)indole, and 5-fluorouracil.

According to one embodiment herein, the silicon nano carrier is used to deliver the anti-cancer drugs. The anticancer drug is 5-fluorouracil. The silicon nano carrier has a pore size of 50 nm.

According to one embodiment herein, the silicon nano carrier is used to deliver a hormone, and wherein the hormone is progesterone.

According to one embodiment herein, the silicon nano carrier is used to deliver anti-fungal drugs in humans. The anti-fungal drugs are selected from a group consisting of clotrimazole, topical nystatin, fluconazole, and topical ketoconazole. The silicon nano carrier is used to treat the fungal skin diseases in humans.

According to another embodiment herein, a silicon nano carrier for delivering the pesticides and the herbicides comprises diatom frustules and a pesticide or a herbicide. The diatom frustule is arranged in a form of a half cylinder. The diatom frustule has a comb-like structure. The pesticide or the herbicide is present inside the diatom frustules. The diatom frustule includes Hannaea arcus and Navicula inflexa. The diatom frustule has a pore size of 1 to 100 nm. The pesticides are selected from a group consisting of an active form of Gallic acid, benzimidazol and conazol, organophosphates, and wherein the herbicides are selected from a group consisting of chloroacetanilide herbicides and anilide herbicides, and wherein the benzimidazol includes albendazole and benomyl, and wherein the conazol includes azaconazole and bromuconazole, wherein the organophosphates include Captan and Diazinon, wherein the chloroacetanilide herbicides includes benzoylpropand flampropand wherein the anilide herbicides include cisanilide and chloranocryl.

According to one embodiment herein, a silicon nano carrier for wastewater treatment comprises a diatom frustules and an absorbent. The diatom frustule is arranged in a form of a half cylinder. The diatom frustule has a comb-like structure. The diatom frustule includes Hannaea arcus and Navicula inflexa. The diatom frustules is an immobilized diatom frustule. The diatom frustule has a pore size of 1 to 100 nm. The absorbent is poly-L-lysine.

According to the embodiments herein, the diatom frustules are comb-like shaped diatom frustules. The diatom frustules in the embodiments herein are Hannaea arcus and Navicula inflexa skeletons. The comb like diatoms frustules act as a nano drug delivery carrier. The nano drug delivery carrier comprises of silicon nano structures of the diatoms. The pore size of the silicon nano structure ranges from 1 to 100 nm. The novel nano carrier described in the embodiments herein is absolutely natural and consists of silicon dioxide (SiO₂) molecules that are arranged in semi cylindrical shape. The semi cylindrical shape provides an optimum space within the diatom frustules where the drug molecules can be loaded. The loaded diatom frustules release the drug molecules at the target site. Due to the porous structure of the diatom frustules, the drug molecules are released slowly, thereby providing a drug delivery mechanism with a controlled release. The comb like diatom frustules in the embodiments herein is in the form of a powder wherein the diatom frustules are sieved diatomaceous earth (DE).

According to an embodiment herein, the nano drug delivery system or a nano silicon vehicle made up of diatom frustules is used for delivering anti cancer drugs. The pore size for delivering the anti cancer drugs is 50 nm. The anti cancer drug is 5-fluorouracil.

According to an embodiment herein, the nano drug delivery system or the diatom drug delivery system is used for delivering various agricultural drugs, pesticides and herbicides. The pore size of the diatom frustules for the delivery of agricultural drugs, pesticides and herbicides is within 1-100 nm. The various pesticides are selected from a group consisting of any active form of Gallic acid, benzimidazol (e.g. albendazole, benomyl), conazol (e.g. azaconazole, bromuconazole), Organophosphate (e.g. Captan and Diazinon), chloroacetanilide herbicides (e.g. benzoylprop, flamprop) and anilide herbicides (e.g. cisanilide and chloranocryl).

According to an embodiment herein, the nano drug delivery system or the diatomaceous drug delivery system is used for a skin treatment. The diatomaceous drug delivery system is used for treating the fungal skin diseases in humans. The fungal drugs that are loaded in the diatom drug delivery vehicle are selected from a group consisting of clotrimazole, topical nystatin, fluconazole, and topical ketoconazole.

According to an embodiment herein, the diatom drug delivery system is used for the removal of hormonal waste from wastewater and industrial sewage. The drug delivery system comprises of diatom frustules in the form of diatomaceous earth sieved particles. The diatom drug delivery system is used in an immobilized form or suspended form or solid-state form from waste treatment system. The immobilized, or suspended or solid-state waste treatment system comprises of diatomaceous earth.

According to another embodiment herein, the diatomaceous drug delivery system can be used for hormone treatment. The diatom drug delivery system is used for delivery of hormones at a target site. The hormone comprises of progesterone.

The embodiments herein provide a nano drug delivery carrier using silicon nano structure of diatoms by pore size ranging from 1 to 100 nm applicable in cancer nano drug delivery. The nano silicon vehicle of maximum 50 nm with applicability of loading anticancer medicine e.g. 5-flouracil, with the particular release time is described herein. The process for production of a nano drug delivery vehicle with the pore sizes of not more than 100 nm. The device characterized in that has functional groups of hydroxyl and has an ability of carrying suitable cancer drugs. The powder having comb-like shaped diatom frustules comprises sieved diatomaceous earth (DE). The agricultural nano vehicle having a pore size ranging from 1 to 100 nm applicable in a pesticide delivery is described herein. The loaded pesticide is any active form of Gallic acid, benzimidazol (e.g. albendazole, benomyl), conazol (e.g. azaconazole, bromuconazole), Organophosphate (e.g. Captan and Diazinon), chloroacetanilide herbicides (e.g. benzoylprop, flamprop) and anilide herbicides (e.g. cisanilide and chloranocryl). An immobilized, suspended, or solid-state waste treatment system comprising diatomaceous earth. The method wherein diatomaceous earth sieved particles is used for removal of hormones from wastewater or industrial sewage. The method wherein diatomaceous earth sieved particles are used for hormone treatment of progesterone. The method wherein nano-silicon structure is used for skin treatment of human fungi. The method wherein the loaded fungicide is any compound like clotrimazole, topical nystatin, fluconazole, and topical ketoconazole.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a schematic pictorial representation of the diatom used as drug delivery system, according to one embodiment herein.

FIG. 2A shows the Light Microscopic Imaging of some grown Diatoms used as drug delivery system, according to one embodiment herein.

FIG. 2B shows the Light Microscopic Imaging of some grown Diatoms used as drug delivery system, according to one embodiment herein.

FIG. 3 shows the pores patterns of Diatoms Navicula inflexa and Hannaea arcus, according to one embodiment herein.

FIG. 4 shows atomic structure of simulated nanosilicon carrier, according to one embodiment herein.

FIG. 5 shows a release pattern of Benomyl measured by NanoDrop in a drug delivery system with a controlled release, according to an embodiment herein.

FIG. 6 shows a release pattern of 5-Florouracil measured by NanoDrop in a drug delivery system with a controlled release, according to an embodiment herein.

FIG. 7 shows release pattern of Progesterone measured by NanoDrop in a drug delivery system with a controlled release, according to an embodiment herein.

FIG. 8 shows Prohibit Transformed Responses in in vivo study of silicon nano structure in a drug delivery system with a controlled release, according to an embodiment herein

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide the use of nano pores of diatom frustules as a new silicon nano-carrier, which is used as a nano drug delivery system. The new nano carrier is absolutely natural and consists of silicon dioxide (SiO₂) molecules that are arranged in a semi cylinder form. Diatoms are a large group of unicellular algae with more than 10,000 species. Diatoms have siliceous cell wall, which is called frustules. Diatoms are vastly expanded and easily available on the Earth. Diatoms have very fine and strong siliceous structure. Therefore, they are suitable for using in industry, nanotechnology and drug delivery. The biosilica in the diatom structure make special nanostructure patterns. In the embodiments herein, a suitable form of frustule from native species of marine diatoms is extracted. The extracted diatom looks like a half cylinder or a semi cylinder. The half cylindrical skeleton of diatom is loaded with different concentrations of various medicines. The medicines are released accordingly. The release of these medicines has been calibrated using appropriate tools. This optimized loaded nano frustules used as enclosed drug delivery systems and the in vivo experiments conducted showed their potency to be used as new nano silicon carrier in pharmaceutical industry.

The drug delivery using diatom frustules according to the embodiments herein can be used in different kind of agricultural applications such as water filtration for agriculture and delivery of agricultural pesticides. The agricultural pesticide can be loaded into the diatom frustules and can be sprayed to the shrubs when exposed to sunlight for a long time with more stability.

Immobilized form of diatom frustules can also be used for delivering various drugs. The immobilized diatom consists of a pure special kind of skeleton in addition to some kind of absorbent. The diatom shells are easy to manipulate when grown on an oxidized silicon surface up to a size of 200 μm in diameter. There are two ways by which the diatom shells can be immobilized: first, a chemical linkage approach using poly-L-lysine and second, involving use of UV-polymerizable epoxy. Controlled etching of the silica structure has been employed to manipulate both the dimensions of the nonporous as well as the pore hierarchy. A simple technique for immobilization of diatom frustules is as follows: silicon wafer coated with polylysine (from 0.01% aqueous solution of polylysine) are used. A drop of cleaned diatom frustule suspension is deposited onto a cleaned and modified silicon wafer. Frustules get settled on the substrate surface exposing either their external surface (concave) or internal surface (convex) forming immobilized frustules. The external and the internal surface have different porous structures. These diatom frustules serve as disposable master where the porous structure is replicated into a polymer.

In some pharmaceutical industries producing hormones, the hormonal waste is dumped into water and soil resources. This is harmful to the living organisms. For this, a special kind of treatment is needed that is capable of collecting and picking up hormonal wastes. So immobilized diatom alone or together with another absorbent can be used that collects or absorbs this kind of waste and makes soil clean.

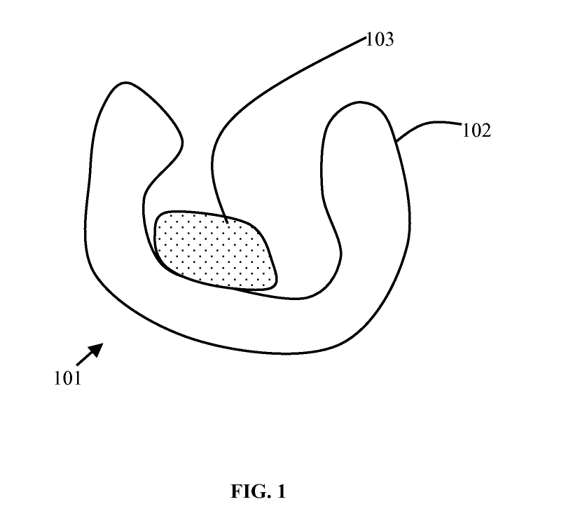

FIG. 1 shows a diagrammatic representation of the diatom used as drug delivery system, according to the embodiments herein. With respect to FIG. 1, the nano drug delivery system 101 comprises of diatom structure 102 with a loaded drug molecule 103. The diatom structure 102 is in the form of a half cylinder. The diatom structure 102 is siliceous in nature with pores. The shape of the diatom structure 102 provides a space for a proper loading of a drug molecule 103 that is released at the delivery site. The pore size is 1 to 100 nm.

Almost any drug can be used or loaded in the diatom structure according to the embodiments herein. There is no limitation in applying various agricultural or medical compounds into the vehicle. However, the release profile should be measured and should be found suitable for the particular application. According to the embodiments herein, any bioactive compound (for human, plants, veterinary domestic or wild animal applications), bioreactors-fermentation tanks-bioremediation, oil spill clean ups, waste management can use the diatom cell wall described in the embodiments herein. The only exception can be injectable forms, as the mentioned vehicle cannot enter into blood stream, or otherwise can cause blood clot formation and most likely death.

### Example 1

### Culture of Diatom Species from Different Region of Iran

Sampling was done from Caspian sea, Jajrod river and Persian Gulf. Samples were cultured using F/2 guilard nutrient medium. This medium has three major ingredients of phosphate, nitrate and silicate. Totally 15 different medium were prepared and samples were cultured into them. The major ingredients of F/2 are shown in table 1.

TABLE 1
showing the major ingredients of F/2 culture medium

| | |
|---|---|
| 1 ml | NaNO₃ (75 g/L dH₂O) |
| 1 ml | NaH₂PO₄•H₂O (5 g/L dH₂O) |
| 1 ml | Na₂SiO₃•9H₂O (30.0 g/L dH₂O) |
| 1 ml | f/2 Trace Metal Solution |
| 0.5 ml | f/2 Vitamin Solution |
| 1 ml | Filtered seawater to |

The ingredients of F/2 Trace Metal Solution are shown in Table 2.

TABLE 2
showing the ingredients of Trace Metal Solution of F/2 culture medium

| | |
|---|---|
| 3.15 g | FeCl₆•3H₂O |
| 4.36 g | Na₂EDTA•2H₂O |
| 1 ml | CuSO₄•4H₂O (9.8 g/L dH2O) |
| 1 ml | Na₂MoO₂•4H₂O (6.3 g/L dH₂O) |
| 1 ml | ZnSO₄•7H₂O (22.0 g/L dH₂O) |
| 1 ml | CoCl₆•2H₂O (10.0 g/L dH₂O) |
| 1 ml | MnCl₆•2H₂O (180.0 g/L dH₂O) |
| 1 ml | Distilled water to |

The ingredients of F/2 Vitamin Solution are listed out in Table 3.

TABLE 3
showing the various ingredients of Vitamin
Solution of F/2 culture medium

| | |
|---|---|
| Vitamin B₁₂ (1 g/L dH₂O) | 1 ml |
| Biotin (0.1 g/L dH₂O) | 10 ml |
| Thiamine HCl | 200 mg |
| Distilled water to | 1 liter |

The various ingredients of Added Booster Solution are shown in Table 4.

TABLE 4
showing the various ingredients of the Added Booster
Solutions of F/2 culture medium

| | |
|---|---|
| Sodium Glutamate | 1.7 g |
| Sodium Glycero-phosphate in 1 liter of DIW | 0.2 g |

The various Antibiotic Solutions of the F/2 culture medium are listed in Table 5 below.

TABLE 5
showing the Antibiotic Solutions of F/2 culture medium

| | |
|---|---|
| Penicillin G+ | 15 g |
| Streptomycin in 250 ml of DIW | 15 g |

The light was prepared using two lamps i.e. yellow lamp of 200 w and white lamps of 50 w. The lamps were placed at a distance of 30 cm from culture containers. This condition was maintained for 24 hours. Air pumps of R1 SHENG, RS-608; CHINA MADE was used to freshen up the air in the liquid culture. Adding 1.5 percent agar to the liquid culture leads to solid cultures.

### Example 2

### Diatom Growth Monitoring

After culturing diatom, with 3 day interval, their growth was monitored. The results are shown in table 6.

TABLE 6
showing the Diatom growth results

| | Date | | | | |
|---|---|---|---|---|---|
| Concentration | Dec. 20, 2005 | Dec. 24, 2005 | Dec. 28, 2005 | Feb. 1, 2006 | Jun. 1, 2005 |
| NaH₂PO₄•H₂O | | | | | |
| 4 gr/l | ++Navicula | ++Navicula | +Cymbella | +Cymbella | +Cymbella |
| | inflexa | inflexa | minuta | minuta | minuta |
| 5 gr/l | ++Navicula | ++Navicula | +Cymbella | ++Cymbella | +Cymbella |
| | inflexa | inflexa | minuta | minuta | minuta |
| 6.5 gr/l | +Cymbella | +Hannaea | +Hannaea | +Cymbella | +Cymbella |
| | minuta | arcus | arcus | minuta | minuta |
| 8 gr/l | ++Hannaea | ++Hannaea | ++Hannaea | +Cymbella | ++Navicula |
| | arcus | arcus | arcus | minuta | inflexa |
| 9.5 gr/l | ++Hannaea | ++Hannaea | +Cymbella | +Cymbella | +Cymbella |
| | arcus | arcus | minuta | minuta | minuta |
| NaNO₃ | | | | | |
| 70 gr/1 | +Navicula | — | — | — | — |
| | inflexa | | | | |
| 75 gr/1 | +Cymbella | +Cymbella | ++Cymbella | ++Cymbella | +++Cymbella |
| | minuta | minuta | minuta | minuta | minuta |
| 85 gr/1 | +Hannaea | +Hannaea | ++Hannaea | Cymbella | Cymbella |
| | arcus | arcus | arcus | minuta | minuta |
| 95 gr/1 | Missed data | Missed data | Missed data | Missed data | Missed data |
| 100 gr/1 | +Hannaea | +Hannaea | +Hannaea | ++Cymbella | ++Cymbella |
| | arcus | arcus | arcus | minuta | minuta |
| Na₂SiO₃•9H₂O | | | | | |
| 20 gr/1 | ++Navicula | ++Navicula | +Cymbella | +Cymbella | ++Navicula |
| | inflexa | inflexa | minuta | minuta | inflexa |
| 30 gr/1 | ++Navicula | ++Navicula | +Cymbella | +Hannaea | ++Navicula |
| | inflexa | inflexa | minuta | arcus | inflexa |
| 40 gr/1 | ++Navicula | ++Hannaea | +Cymbella | +Cymbella | ++Hannaea |
| | inflexa | arcus | minuta | minuta | arcus |
| 50 gr/1 | ++Navicula | ++Navicula | ++Cymbella | ++Navicula | ++Navicula |
| | inflexa | inflexa | minuta | inflexa | inflexa |
| 60 gr/1 | ++Navicula | ++Navicula | ++Navicula | ++Cymbella | ++Navicula |
| | inflexa | inflexa | inflexa | minuta | inflexa |

* +shows usual growth and ++shows excellent growth

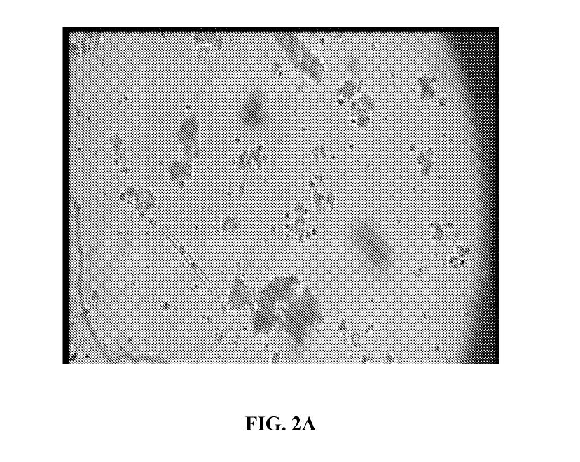

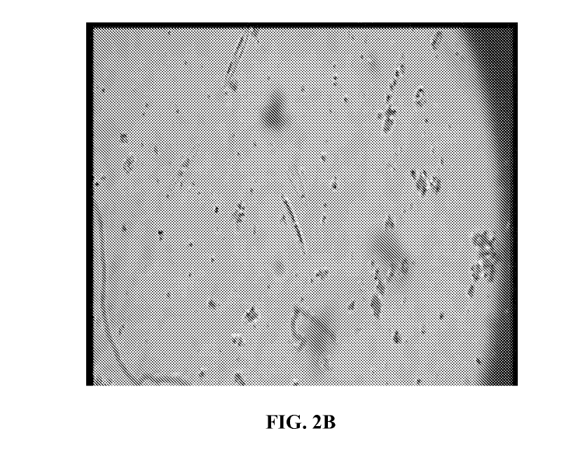

Result has shown that different concentration of nitrate, phosphate and silicate can affect the diatom growth. For example table shows that nitrate stimulate the growth of Cymbella minuta and small concentrations of phosphate stimulate the growth of Cymbella minuta. Light microscopic imaging of some grown diatoms are shown in FIG. 2. FIG. 2A and FIG. 2B shows the Light Microscopic Imaging of some grown Diatoms. The diatoms are in a desired form as required for use. With respect to FIG. 2A and FIG. 2B, it can be figured out that the predominantly grown species of Diatoms in the nutritive medium are pennate diatoms (Pennales), which are bilaterally symmetric.

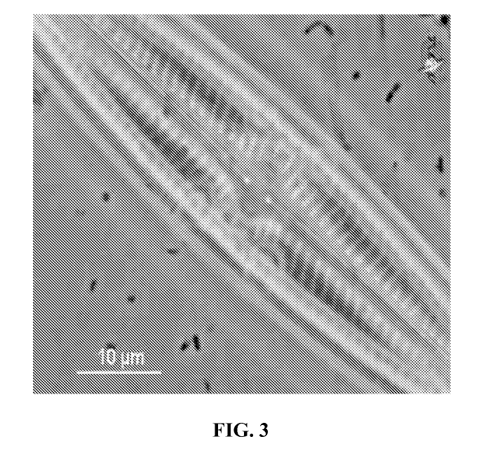

Also the results show that Hannaea arcus and Navicula inflexa are predominant diatom species of Iran that according to their frustules pattern of uncentric diatoms, they can be used for nanocarrier design. Their pores patterns are shown in FIG. 3. FIG. 3 shows the pores patterns of Diatoms Navicula inflexa and Hannaea arcus, according to the embodiment herein. Navicula inflexa and Hannaea arcus are the two common types of Diatoms in Iran and grow too fast in nutritive medium. These Diatoms are used as a model for simulation part as well as for other experimental steps.

It was predicted that some special kind of frustule with parallel costa could be used for drug delivery system. Navicula inflexa and Hannaea arcus have this kind of frustules but any other kind of diatom with this special kind of cell wall can be used in drug delivery as well.

As can be seen in FIG. 3, the diatom pores are very regular and evenly ordered. They have regular size of nanometer (varying from 0.5 to 5 nm) and they are species specific. The pore size of a diatom affects the loading and release profile of a drug. The smaller the pores a longer release is expected.

### Example 3

### Simulation of Nanostructure and Evaluation of its Infarction with Target Compounds

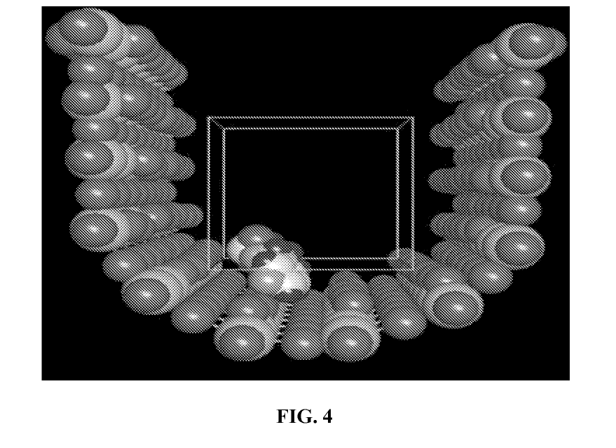

Designing and drawing of silicon nanostructure was done in Chemdraw Ultra (Cambridge, ChemOffice Suite, 8.0.3, 2004) and Chemwindow (Bio-Rad Labaoratories, 6.5, 2005). To understand the behavior of the molecules in silicon nanochamber, the Arguslab molecular docking Software, (4.01, 2005) was applied to obtain interaction energy (IE) measures between the bioactive compound's molecule and the silicon surface. IE for the molecule was calculated as if there were no other molecules in the chamber. The units for IE are Kcal/mol. The grid resolution was 0.4 and augment root node was applied false. The binding site box for docking was of dimension of 150×150×150 angstrom, as it was felt these dimensions gave the best position for the molecule if no other molecules are present. The smaller the overall dimension of the binding site, the larger IE, so molecules with lower index interact more strongly with surface hydroxyl groups. Simulation has shown that the surface morphology of the diatom frustules must follow some 3D structure. FIG. 4 shows the 3D structure of the diatom frustules. FIG. 4 shows atomic structure of simulated nano-silicon carrier, according to the embodiments herein. With respect to FIG. 4, the basic arrangement of silica nano-carrier from Diatoms frustules is depicted. FIG. 4 shows that silica atoms are not directly connected to each other using covalence bond but instead they are connected to Oxygen atoms. The rectangle in FIG. 4 shows the typical DOCK space where the drug can be loaded. Its dimension is optional and could be different. The black ones are oxygen molecules and the white ones are silica molecules. The molecule placed in the centre inside the rectangular space is the drug molecule.

The basic component of the molecules is SiO₂. The structure shows a basic atomic arrangement of nano silicon, similar to what can be seen in carbon nanotube. The major difference between these two structures is that silicon nanostructure is composed of silicon and oxygen, while carbon nanotube is composed of only carbon atoms. Some docked molecules are shown in table 6.

TABLE 6
list of docked molecules into silicon nanochamber

Acarbose
Acetaminophen
Acetanilide
Acetazolamide
Acetylsalicylic acid
Allopurinol
Alosetron
Alphaprodine
Aminogluthetimide
Amodiaquine
Amthamine
Aprepitant
Aromatase inhibitor
Balsalazide
Benzamides sulpiride
Benomyl
Benzylhydrazine
Benzylpenicillin
Bezafibrate
Buclizine
Caffeine
Carbendazim
Carbutamide
Carmustine
Chlorambucil.A
Chlorhexidine
Chlorobutanol
Ciproflaxacin
Clemastine
Clobazam
Clomethiazole
Cyclaradine
Cyclophosphaide
Decarbonylase inhibitor
Diflunisal
Diperodon
Diphenhydramine
Diphenoxylate
Diphenydramine
Diphenylbutylpiper
Diphenylpyraline
Domperidone
Dopamine
Doxylamine
Ephedrine
Epinephrine
Ethanolamine
Etomidate
Eugenol
Fadrazole
Felodipine
Flufenamic acid
Fluorouracil
Flurandrenolone
Furazolidone.A
Fenbuconazole
Gabapentin
Gallic acid
Histamine
Hordenine
Hydralazine
Hexachlorobenzene
Hydroxyzine
Hyoscyamine
Ibuprofen
Indomethacin
Isogramine
isoleucine
Isoproterexol
Ketorolac
Khellin
Lamotrigine
Lansoprazole
Lisoleucine
Lomustine
Lysine
Marinol
Meperidine
Mercaptopurine
Mescaline
Metformin
Methadone
Methantheline
Methicilin
Methoxamine
Methyldopahydrazine
Metronidazole
Metronidazole
Metyrapone
Misonidazole
Mitomycin C
Muzolinine
Nabilone
Nafoxidine
Natamycin
Natamycin
Nicolsamide
Nicotine
Nifurtimox
Nizatidine
Nystatin
Ofurace
Ofloxacin
Omeprazole
Oxamniquine
Oxaprozine
Pentamidine
Phenacetin
Phenmetrazine
Phenoxymethylpenicilin
Phenphormin
Phenylephrine
Phosphor amide
Polymyxin B Sulfate
Pramoxine
Praziquantal
Procaine
Progestrone
Pyrantel
Quinacrine
Razoxane
Ritodrine
Rosiglitazone
Roxatidine
Salicylamid
Selenazofurin
Sulfaalazine
Sulfacytine
Sulfamerazine
Sulfamethazine
Sulfamethizole
Sulfamethoxazole
Sulfanilamide
Sulinpyrazone
Terfenadine
Thiabendazole
Thiabedazole
Thiaisoleucine
Thialysine
Tiazofurin
Ticlopidine
Thiophanate-methyl
Tilorone
Tocainide
Tolazamide
Triplennamine
Valnoctamide
Valporic acid
Vicuprofen
Vigabartin
Zidovudine
Zopicolone
4-hydroxyandrostene
5-Hydroxytryptamine
3,4-epoxide
2′3′-Dideoxyinosine
2′,3′-dideoxy-2′,3′-didehydrocytidine(d4C)
1-(5-hydroxymethyl-2-furyl)-9H-pyrido(3,4-b)indole
5-fluorouracil

Dock results for some major compound are shown in table 7:

| | Loading | |
|---|---|---|
| Name | value | Bioactivity |
| Gallic acid | −4.98 | Antifungal properties in various crops |
| Podophyllotoxin | −3.7 | Podophyllotoxin is the pharmacological precursor for the |
| | | important anti-cancer drug |
| Progesterone | −4.41 | |
| Ofloxacin | −4.68 | Ofloxacin is bactericidal and its mode of action depends on |
| | | blocking of bacterial DNA replication |
| Cobazam | −4.45 | Clobazam is a barbiturate used in combination with |
| | | acetaminophen or aspirin and caffeine for its sedative and |
| | | relaxant effects in the treatment of tension headaches, |
| | | migraines, and pain |
| Piroxicon | −4.42 | Feldene(piroxican) is a non-steroidal anti-inflammatory |
| Ciproflaxacin | −4.41 | A broad-spectrum antimicrobial carboxyfluoroquinoline |
| Terfenadine | −4.41 | Terfenadine is an antihistamine formerly used for the |
| | | treatment of allergic conditions |
| Fadrazole | −4.39 | Anticancer |
| Ketotifene | −4.38 | A cycloheptathiophene blocker of histamine H1 receptors |
| | | and release of inflammatory mediators. It has been proposed |
| | | for the treatment of asthma, rhinitis, skin allergies, and |
| | | anaphylaxis |
| Amodiaquine | −4.33 | A 4-aminoquinoquinoline compound with anti- |
| | | inflammatory properties |
| Nafoxidine | −4.33 | An estrogen antagonist that has been used as an |
| | | antineoplastic |
| MitomycinC | −4.32 | Mitomycin-C is an anti-cancer chemotherapy drug |
| Hydralazine | −4.31 | is a direct-acting smooth muscle relaxant used to treat |
| | | hypertension by acting as a vasodilator primarily in arteries |
| | | and arterioles |
| Phenmetrazine | −4.31 | Phenmetrazine is a sympathomimetic drug used primarily as |
| | | an appetite depressant |
| Dexrazoxane | −4.27 | Antineoplastic Agents Cardiovascular Agents |
| Diphenylpyraline | −4.27 | Antihistamine |
| lamotrigine | −4.26 | an anticonvulsant drug used in the treatment of epilepsy and |
| | | bipolar disorder |
| Indomethacin | −4.24 | an anthelmintic used in most schistosome and many cestode |
| | | infestations |
| Benomyl | −4.85 | Systemic fungicide used against various pathogen of crops |
| Thiabendazole | −4.23 | Thiabendazole is also a chelating agent |
| Tolcainide | −4.23 | Used to treat Cardiac Arrhythmias |
| Pyrantel | −4.2 | is used as a deworming agent in the treatment of hookworms |
| | | (all species) and roundworms |
| Buclizine | −4.2 | An antivertigo/antiemetic agent. |
| Sulfamethazine | −4.19 | A sulfanilamide anti-infective agent. It has a spectrum of |
| | | antimicrobial action similar to other sulfonamides |
| Acetaminophen | −4.19 | Anti pain |
| Zidovudine | −4.19 | Zidovudine was the first drug approved for the treatment of |
| | | AIDS and HIV infection |
| Benzolpenicilin | −4.17 | effective against most gram positive bacteria and against |
| | | gram-negative cocci |
| Allopurinol | −4.17 | Allopurinol is a drug used primarily to treat conditions |
| | | arising from excess uric acid in blood plasma |
| Sulfacytine | −4.17 | synthetic bacteriostatic antibiotics with a wide spectrum |
| | | against most gram-positive and many gram-negative |
| | | organisms |
| Furazolidone | −4.17 | Antibacterial |
| Sulinpyrazone | −4.16 | uricosuric medication used to treat gout |
| Metyrapone | −4.15 | It is used as a test of the feedback hypothalamic-pituitary |
| | | mechanism in the diagnosis of cushing syndrome |
| Sulfamethizole | −4.14 | The sulfonamides are synthetic bacteriostatic antibiotics |
| carbutamide | −4.14 | anti-diabetic drug |
| Triplennamine | −4.14 | Anti-Allergic Agents Ethylenediamine Derivatives |
| | | Histamine |
| Sulfamerazine | −4.13 | sulfonamide antibacterial |
| fluorouracil | −4.3 | antineoplastic anti-metabolite |
| Isoniazide | −4.1 | antibiotic medicine used to prevent or treat tuberculosis |
| Oxaminoquine | −4.09 | anthelmintic with schistosomicidal activity against |
| | | Schistosoma mansoni |
| Doxylamine | −4.09 | Doxylamine |
| Sulfamethoxazole | −4.08 | bacteriostatic antibiotic |
| Methoxamine | −4.07 | Hypertensive |
| Ritodrinel | −4.07 | Adrenergic beta-agonist used to control premature labor |
| Acetanilide | −4.05 | Not DRUG |
| Ibuprofen | −4.05 | |
| Diphenhydramine | −4.05 | antihistamine of the ethanolamine class |
| Tilorone | −4.05 | orally active interferon inducer |
| Albuterol | −4.04 | a bronchodilator that relaxes muscles |
| Hydroxyzine | −4.04 | first-generation antihistamine, |
| Sulfanilamide | −4.03 | sulfonamide antibiotic |
| Metronidazole | −4.03 | used mainly in the treatment of infections caused by |
| | | susceptible organisms, particularly anaerobic bacteria and |
| | | protozoa |
| Gabapentin | −4.02 | used as an anticonvulsant to treat partial seizures, |
| | | amyotrophic lateral sclerosis (ALS), and painful |
| | | neuropathies |
| Phenacetin | −4.01 | analgesic without anti-inflammatory properties |

According to embodiments herein, the diatom structure and drug loading capacity can vary for different kind of medicines. For example for anticancer Fluorouracil (5-FU or f5U) a master mix of 50 mg/ml was prepared. 0.8 ml of this master mix was added to 200 ml pure ethanol. 0.6 g diatomaceous earth (diatom cell wall) was added to this mixture and was left to be stirred for 3 hours with 200 rpm. Finally after filtration, diatom cell wall was washed once with methanol. 12 hours later at room temperature diatom cell wall were dried and loaded with Fluorouracil (5-FU or f5U).

The results of Docking interaction energies shown that different compounds have different interaction energies that affect their binding to nano silicon carrier. This means that those with higher interaction energy attach into nano silicon carrier more tightly and may not get away of it and vice versa. This property was used to choose the target. For example for elimination of hormonal contamination purpose, the molecules that tightly bind to nano silicon and do not leave it were chosen. In vitro study confirms these results.

### Example 4

### In Vitro Nano Spectrophotometer

Evaluation of Gallic Acid Release by Nano Drop Spectrophotometer:

Final concentration of 200 mg/ml of Gallic acid in Methanol/Diatomaceous Earth was prepared. To ensure that none of the Gallic acid was left on the outer surface of nano silicon, it washed by Methanol. After his, its release in water by interval of 2 hours was measured at the wavelength of 270 nm. Results have shown that all optical density (ODs) measures were zero and none of the Gallic acid has been released in water. These results are in accordance with Dock results, because the Gallic acid has the highest interaction energy among the compounds.

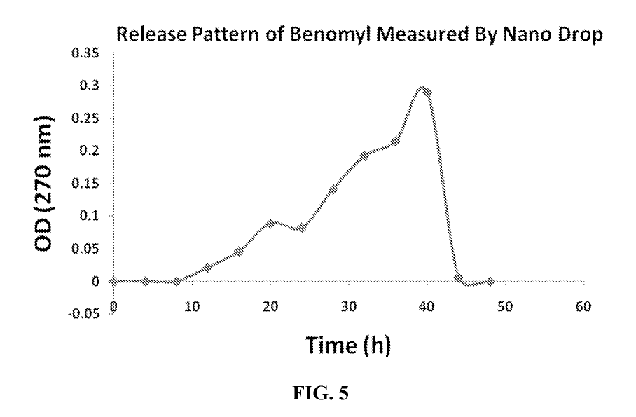

Evaluation of Benomyl fungicide release by nano Drop Spectrophotometer: Material and methods were identically same as Gallic Acid. After measuring all ODs, release calibration curve was drawn. FIG. 5 shows release pattern of Benomyl measured by NanoDrop. With respect to FIG. 5, it is seen that the Benomyl releases after 12 hours of loading and gets to its peak release after 40 hours and the release decreases sharply. This is a kind of controlled release.

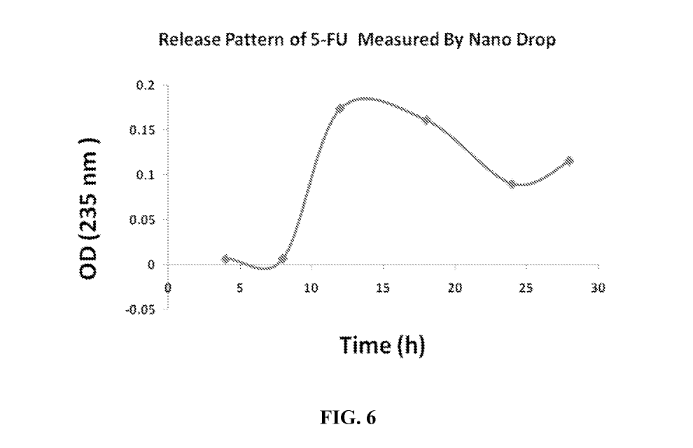

5-flouracil release pattern by nano Drop Spectrophotometer was evaluated. FIG. 6 shows release pattern of 5-Florouracil measured by NanoDrop. With respect to FIG. 6, it can be seen that the drug 5-Florouracil releases after 8 hours from the loading starts and 16 hours after gets peak release and then fall of gradually. This is a kind of controlled release.

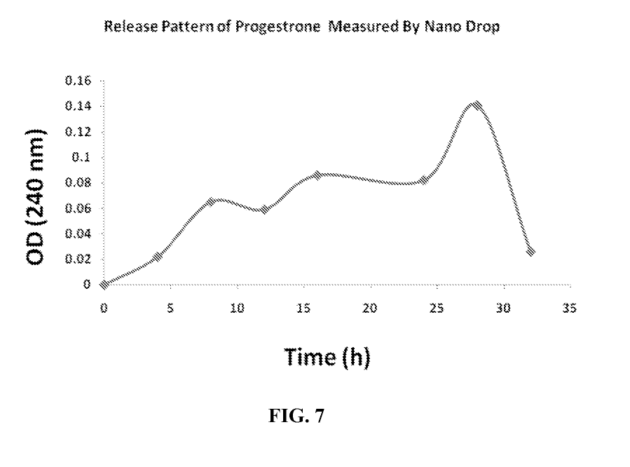

Progesterone release pattern by nano Drop Spectrophotometer was evaluated. FIG. 7 shows release pattern of Progesterone measured by NanoDrop. With respect to FIG. 7, the drug shows a release after 4 hours from the loading began and 28 hours later has its peak, then fall off suddenly.

### Example 5

In vivo study: In vivo analysis of silicon-nanostructure using Artimia salina was done to make sure that silicon-nanostructure has no toxic effect on living cells. Suspension of salt water Diatomaceous earth in following concentration was prepared. 200 ml of this suspension was poured in every well of 96 well plates and 10 Artimia salina was added to every well. The data is shown in table 8 below. After 14 hours the number of dead Artimia salina were counted and using spss 14 software was analyzed. The probit regression test was used by 95% significant level.

TABLE 8
In vivo analysis of silicon-nanostructure using Artimia salina

| | Repeat | | |
|---|---|---|---|
| g/mlμ concentration | 1 | 2 | 3 |
| 4000 | 3 | 3 | 4 |
| 1000 | 2 | 4 | 3 |
| 500 | 2 | 2 | 0 |
| 250 | 2 | 4 | 0 |
| 125 | 2 | 1 | 1 |
| Control (salt water) | 0 | 0 | 0 |

The Probit Analysis Data is Shown Below:

PROBIT ANALYSIS

DATA Information
21 unweighted cases accepted.
0 cases rejected because of missing data.
3 cases are in the control group.
MODEL Information
ONLY Normal Sigmoid is requested.
Parameter estimates converged after 12 iterations.
Optimal solution found.
Parameter Estimates (PROBIT model: (PROBIT (p)) = Intercept + BX):

| | Regression Coeff. | Standard Error | Coeff./S.E. |
|---|---|---|---|
| VAR00001 | .00017 | .00007 | 2.42722 |

| Intercept | Standard Error | Intercept/S.E. |
|---|---|---|
| −1.07667 | .13780 | −7.81349 |

Pearson Goodness-of-Fit Chi Square = 25.340 DF = 19 P = .150
Since Goodness-of-Fit Chi square is significant, a heterogeneity factor is used in the
calculation of confidence limits.
Observed and Expected Frequencies

| | Number of | Observed | Expected | | |
|---|---|---|---|---|---|
| VAR00001 | Subjects | Responses | Responses | Residual | Prob |
| 4000.00 | 10.0 | 5.0 | 3.506 | 1.494 | .35060 |
| 4000.00 | 10.0 | 2.0 | 3.506 | −1.506 | .35060 |
| 4000.00 | 10.0 | 1.0 | 3.506 | −2.506 | .35060 |
| 2000.00 | 10.0 | 3.0 | 2.326 | .674 | .23264 |
| 2000.00 | 10.0 | 3.0 | 2.326 | .674 | .23264 |
| 2000.00 | 10.0 | 4.0 | 2.326 | 1.674 | .23264 |
| 1000.00 | 10.0 | 2.0 | 1.831 | .169 | .18315 |
| 1000.00 | 10.0 | 4.0 | 1.831 | 2.169 | .18315 |
| 1000.00 | 10.0 | 3.0 | 1.831 | 1.169 | .18315 |
| 500.00 | 10.0 | 2.0 | 1.611 | .389 | .16108 |
| 500.00 | 10.0 | 2.0 | 1.611 | .389 | .16108 |
| 500.00 | 10.0 | .0 | 1.611 | −1.611 | .16108 |
| 250.00 | 10.0 | 2.0 | 1.507 | .493 | .15072 |
| 250.00 | 10.0 | 4.0 | 1.507 | 2.493 | .15072 |
| 250.00 | 10.0 | .0 | 1.507 | −1.507 | .15072 |
| 125.00 | 10.0 | 2.0 | 1.457 | .543 | .14571 |
| 125.00 | 10.0 | 1.0 | 1.457 | −.457 | .14571 |
| 125.00 | 10.0 | 1.0 | 1.457 | −.457 | .14571 |
| .00 | 10.0 | .0 | 1.408 | −1.408 | .14081 |
| .00 | 10.0 | .0 | 1.408 | −1.408 | .14081 |
| .00 | 10.0 | .0 | 1.408 | −1.408 | .14081 |

Confidence Limits for Effective VAR00001
95% Confidence Limits

| Prob | VAR00001 | Lower | Upper |
|---|---|---|---|
| .01 | −7213.53713 | −2048946.0687 | −2839.46216 |

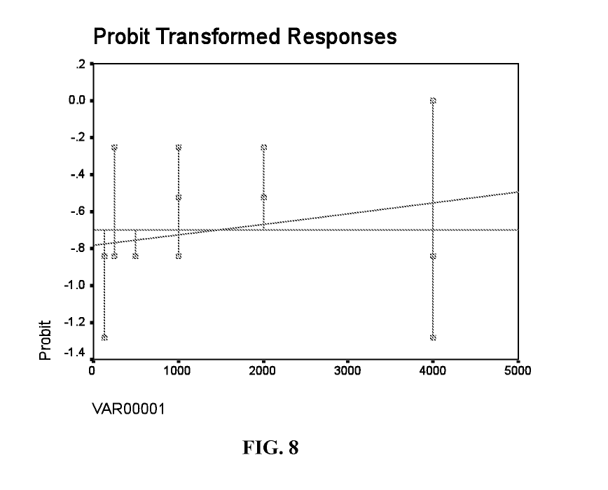

FIG. 8 shows Prohibit Transformed Responses in in vivo study. With respect to FIG. 8, it can be confirmed that the silicon nanostructure has no toxic effect on living cell.

### Example 6

Thin Layer Chromatography: using thin layer chromatography release pattern of studied compounds were evaluated. To run a TLC, the following procedure was carried out:

A small spot of the drug or the frustules containing the medicine was applied to a plate, about 1.5 centimeters from the bottom edge. The solvent was allowed to completely evaporate off. The solvent used was methanol. A small amount of an appropriate solvent eluent was poured into a separation chamber to a depth of less than 1 centimeter. A strip of filter paper was put into the chamber, so that its bottom touched the solvent, and the paper lied on the chamber wall and reached almost to the top of the container. The container was closed with a cover glass or any other lid and was left for a few minutes to let the solvent vapors ascend the filter paper and saturate the air in the chamber. The TLC plate was then placed in the chamber so that the spot(s) of the sample did not touch the surface of the eluent in the chamber. The lid was closed. The solvent moved up the plate by capillary action, reached the sample mixture and carried it up the plate (elutes the sample). When the solvent front reached no higher than the top of the filter paper in the chamber, the plate was removed and dried. In case where the spots were colorless, they were observed and located under UV light.

It was observed that the different medicines in the sample mixture travel at different rates due to the differences in their attraction to the stationary phase, and because of differences in solubility in the solvent. By changing the solvent, or perhaps using a mixture, the separation of the components was adjusted.

A method of preparing a nano silicon carrier, according to the embodiments herein, includes conducting literature searches and getting information about possibility of innovation and silicon nano-carrier structure. Further, performing sampling of the native diatoms in different area of Iran. Culturing the diatoms and optimizing their growth conditions using F/2 medium. Performing Light microscopy imaging of grown diatoms and selecting the desired species for next step. Performing simulation of silicon nano-carrier structure using variety of softwares including Chemoffice. Optimizing the silicon nanocarrier using Molecular Mechanics methods. Selecting different molecule for Dock or for Loading virtual test. Docking different molecules into silicon nanochamber and choosing the best of them with most negative interactive energies. Performing in vitro analysis of the selected drugs using Nano Spectrophotometer. Performing in vivo bioactivity analysis of silicon-nanostructure using Artimia salina.

Diatoms are a large group of unicellular algae (more than 10,000 species). They have siliceous cell wall which is called frustules. Diatoms are vastly expanded and easily available on the Earth. Diatoms have very fine and strong siliceous structure; therefore they are suitable for using in industry, nanotechnology and drug delivery as their biosilica make special nanostructure patterns. In this invention the suitable form of Frustule, extracted from native species of marine diatoms that looks like a half of cylinder. This pure skeleton loaded with Different concentration of various medicines and release of them has calibrated using appropriate tools. This optimized loaded nano frustules used as enclosed drug delivery systems and in vivo experiments showed their potency to be used as new nano silicon carrier in pharmaceutical industry.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A silicon nano carrier comprising:
a diatom frustule, and wherein the diatom frustule is in a form of a half cylinder and wherein the diatom frustule has a comb-like structure; and
a drug, and wherein the drug is present inside the diatom frustule and wherein the drug is released at a target site.

2. The silicon nano carrier according to claim 1, wherein the diatom frustule includes Hannaea arcus and Navicula inflexa.

3. The silicon nano carrier according to claim 1, wherein the diatom frustule has a pore size of 1 to 100 nm.

4. The silicon nano carrier according to claim 1, wherein the diatom frustule is in a powder form.

5. The silicon nano carrier according to claim 1, wherein the diatom frustule is sieved diatomaceous earth.

6. The silicon nano carrier according to claim 1, wherein the silicon nano carrier is used as a drug delivery system, and wherein the silicon nano carrier delivers a drug in a controlled release manner.

7. The silicon nano carrier according to claim 1, wherein the drug is selected from a group comprising of acarbose, acetaminophen, acetanilide, acetazolamide, acetylsalicylic acid, allopurinol, alosetron, alphaprodine, aminogluthetimide, amodiaquine, amthamine, aprepitant, aromatase inhibitor, balsalazide, benzamides sulpiride, benomyl, benzylhydrazine, benzylpenicillin, bezafibrate, buclizine, caffeine, carbendazim, carbutamide, carmustine, chlorambucil.A, chlorhexidine, chlorobutanol, ciprofloxacin, clemastine, clobazam, clomethiazole, cyclaradine, cyclophosphaide, decarbonylase inhibitor, diflunisal, diperodon, diphenhydramine, diphenoxylate, diphenydramine, diphenylbutylpiper, diphenylpyraline, domperidone, dopamine, doxylamine, ephedrine, epinephrine, ethanolamine, etomidate, eugenol, fadrazole, felodipine, flufenamic acid, fluorouracil, flurandrenolone, furazolidone.A, fenbuconazole, gabapentin, gallic acid, histamine, hordenine, hydralazine, hexachlorobenzene, hydroxyzine, hyoscyamine, ibuprofen, indomethacin, isogramine, isoleucine, isoproterexol, ketorolac, khellin, lamotrigine, lansoprazole, lisoleucine, lomustine, lysine, marinol, meperidine, mercaptopurine, mescaline, metformin, methadone, methantheline, methicilin, methoxamine, methyldopahydrazine, metronidazole, metyrapone, misonidazole, mitomycin C, muzolinine, nabilone, nafoxidine, natamycin, natamycin, nicolsamide, nicotine, nifurtimox, nizatidine, nystatin, ofurace, ofloxacin, omeprazole, oxamniquine, oxaprozine, pentamidine, phenacetin, phenmetrazine, phenoxymethylpenicilin, phenphormin, phenylephrine, phosphor amide, polymyxin B sulphate, pramoxine, praziquantal, procaine, progesterone, pyrantel, quinacrine, razoxane, ritodrine, rosiglitazone, roxatidine, salicylamid, selenazofurin, sulfaalazine, sulfacytine, sulfamerazine, sulfamethazine, sulfamethizole, sulfamethoxazole, sulphanilamide, sulinpyrazone, terfenadine, thiabendazole, thiabedazole, thiaisoleucine, thialysine, tiazofurin, ticlopidine, thiophanate-methyl, tilorone, tocamide, tolazamide, triplennamine, valnoctamide, valporic acid, vicuprofen, vigabartin, zidovudine, zopicolone, 4-hydroxyandrostene, 5-Hydroxytryptamine, 3,4-epoxide, 2′3′-Dideoxyinosine, 2′,3′-dideoxy-2′,3′-didehydrocytidine(d4C),1-(5-hydroxymethyl-2-furyl)-9H-pyrido(3,4-b)indole, and 5-fluorouracil.

8. The silicon nano carrier according to claim 1, wherein the silicon nano carrier is used to deliver anti-cancer drugs, and wherein the anticancer drug is 5-fluorouracil, and wherein the silicon nano carrier has a pore size of 50 nm.

9. The silicon nano carrier according to claim 1, wherein the silicon nano carrier is used to deliver a hormone, wherein the hormone is progesterone.

10. The silicon nano carrier according to claim 1, wherein the silicon nano carrier is used to deliver anti-fungal drugs in humans, wherein the anti-fungal drugs are selected from a group consisting of clotrimazole, topical nystatin, fluconazole, and topical ketoconazole, wherein the silicon nano carrier is used to treat fungal skin diseases in humans.

11. A silicon nano carrier for delivering pesticides and herbicides comprising:
a diatom frustule, and wherein the diatom frustule is in a form of a half cylinder and wherein the diatom frustule has a comb-like shape; and
a pesticide or a herbicide, wherein the pesticide or the herbicide is present inside the diatom frustules.

12. The silicon nano carrier according to claim 11, wherein the diatom frustule includes Hannaea arcus and Navicula inflexa.

13. The silicon nano carrier according to claim 11, wherein the diatom frustule has a pore size of 1 to 100 nm.

14. The silicon nano carrier according to claim 11, wherein the pesticides are selected from a group consisting of an active form of Gallic acid, benzimidazol and conazol, organophosphates, and wherein the herbicides are selected from a group consisting of chloroacetanilide herbicides and anilide herbicides, and wherein the benzimidazol includes albendazole and benomyl, and wherein the conazol includes azaconazole and bromuconazole, and wherein the organophosphates include Captan and Diazinon, and wherein the chloroacetanilide herbicides includes benzoylpropand flamprop and wherein the anilide herbicides include cisanilide and chloranocryl.

15. A silicon nano carrier for waste water treatment comprising:
a diatom frustule, wherein the diatom frustule is in a form of a half cylinder and wherein the diatom frustule has a comb-like shape; and
an absorbent.

16. The silicon nano carrier according to claim 15, wherein the diatom frustule includes Hannaea arcus and Navicula inflexa.

17. The silicon nano carrier according to claim 15, wherein the diatom frustule is an immobilized diatom frustule.

18. The silicon nano carrier according to claim 15, wherein the diatom frustule has a pore size of 1 to 100 nm.

19. The silicon nano carrier according to claim 15, wherein the absorbent is poly-L-lysine.
